(19) United States
(12) Patent Application Publication
(10) Pub. No.: US 2020/0318040 A1
(43) Pub. Date: Oct. 8, 2020

(54) SYSTEM FOR THE PROPAGATION OF YEAST AND ADAPTATION FOR SECONDARY FERMENTATION IN THE PRODUCTION OF SPARKLING WINES

(71) Applicant: INBIOLEV, S.L., Aizoain (Navarra) (ES)

(72) Inventor: David Garcia Yoldi, Aizoain (ES)

(21) Appl. No.: 16/304,813

(22) PCT Filed: May 26, 2017
(86) PCT No.: PCT/ES2017/070360

§ 371 Date: Nov. 27, 2018

(30) Foreign Application Priority Data
May 27, 2016 (ES) P201630695

(51) Int. Cl. C12G 1/06; C12M 1/00; C12M 1/06; C12M 1/34

(52) U.S. Cl. CPC C12G 1/06; C12M 43/02; C12M 29/00; C12M 41/12; C12M 27/02

(57) ABSTRACT

The invention relates to a system for the propagation of yeast and adaptation to ethanol in a sustained manner using a bioreactor (1) which is programmed automatically and in which the propagation and adaptation to ethanol, pH and temperature is carried out, for a secondary fermentation in the tirage of Cava, Champagne and sparkling wines in general. The bioreactor (1) includes a motor (6) with stirring blades, and a temperature-control system comprising a sleeve and an electrical resistor, said bioreactor (1) being associated with a mixing tank (2) which houses the liquor. In addition, a programmed pump (4) is provided between the bioreactor (1) and the mixing tank (2), as well as a programmable automaton with a control panel (5) for automating the process.

### OBJECT OF THE INVENTION

The present invention refers to a system of propagating “pre-ferment” for the second fermentation in the production or elaboration of sparkling wines, being applicable in the tirage of cavas or champagne and in general in sparkling wines.

The invention consists of a first phase only of yeast propagation in oxidative phase without adaptation to ethanol so that in a second phase the sustained and programmed adaptation to ethanol is generated.

Both the preliminary cellular process and the adaptation of these yeasts to the ethanol take place in an automatically programmed bioreactor, in which apart from propagating the yeasts, it adapts to the ethanol, to the pH and to the temperature.

### BACKGROUND TO THE INVENTION

Traditionally, the propagation of yeasts for second fermentation in the generation of sparkling wines is done by adding wine from the beginning of the process and in an imprecise manner, thus the yeast adaptation process is not very smooth, is harmful to the physiology of the yeasts, no sustainment, producing cell damage, with low viability, resulting results yeasts which are not very healthy, logically impacting on the low quality of the sparkling wines obtained.

Traditional systems require the use of high quantity yeasts, so the application of the first propagation process described in the present report represents significant economic savings in yeasts.

Furthermore, in traditional systems, the process is carried out without any type of automatism, with no possibility of adapting precisely, in a fully controlled manner and automatically the second fermentation yeasts to the temperature, pH and ethanol.

### DESCRIPTION OF THE INVENTION

The system which is advocated resolves in a fully satisfactory manner the problem described above in each of the aspects commented.

To do so, it is based on propagating the yeasts without adding wine, so as to obtain sequentially an adaptation of the yeasts in a manner that is smooth, sustained and programmed with the least possible cell damage, offering optimum viability and in short a better quality of resulting sparkling wines.

In addition, using the system of the invention the process is undertaken in a planned and automatic way without the oenologist having to intervene as is required traditionally, allowing gradual adaptation to the temperature and pH and of course to the ethanol.

More specifically, the system of the invention is based on the combination of a bioreactor and a mixing tank, mounted on a frame, with a common programmed pump and a control panel or system which automates the process.

With regard to the bioreactor, it is equipped with a motor with stirring blades as well as air diffusers attached to air filters, where the smallest is 0.01 microns, so sterile air enters the reactor, while the filters are attached via the appropriate piping to a compressor.

The bioreactor also has a temperature control system via cooling chambers of cooling liquid or glycol water, the entry of which is controlled by electro-valve, with resistances inside the bioreactor itself which complement the temperature control chambers.

With regard to the control panel, a frequency converter of the motor is controlled using an automaton, enabling modification of both the motor revolutions and oxygenation using a flow sensor, allowing entry of the necessary quantity of oxygen to each previously programmed phase for the yeasts, while also controlling the electro-valve and resistance for programmed temperature and pump control between the bioreactor and the mixing tank.

In accordance with these characteristics, the propagation and adaptation process of the yeasts for second fermentation in the generation of sparkling wines is as follows:
The bioreactor is filled to half its volume, with the ideal medium for the yeast propagation without the presence of ethanol.
The mixing tank is filled with the liquor (wine, sugar, nutritive elements and/or yeast protectors).
The yeast is activated outside the bioreactor and once activated it is added to the bioreactor.
The process commences with a first programme of the controller, which corresponds to yeast propagation, a process which lasts one day and in which only the bioreactor is involved. At this stage of the process, the conditions of revolutions per minute of the mixing motor are set in the corresponding programme, as well as the flow sensor conditions for the entry of filtered air and temperature conditions.
Once the yeast propagation has passed, automatically the bioreactor enters a second programme, changing the conditions of revolutions per minute, air inlet and temperature, and activating the mixer and the pump to add to the bioreactor the quantities defined during the time the liquor is in the mixing tank. This facilitates better adaptation and greater viability of the yeasts in the adaptation to ethanol, a process that lasts one day.
Once the second programme defined in the previous point has passed, a third programme is activated where once again the conditions of revolutions per minute of the motor, temperature and air inlet change, and liquor continues to enter the mixing tank steadily. This process lasts 1 day.
After this third programme, the yeasts have now been propagated and adapted to the ethanol, pH and temperature, all having been done automatically, by which time the winemaker can use them for the second fermentation tirage. As the tirage is normally expected to take 5 more days, following the 3 days pre-fermenting, during these 5 days the bioreactor moves into a fourth programme where once again the conditions of revolutions per minute of the motor, air inlet and temperature change. At this stage, the mixing tank has emptied and stopped just like the corresponding programmed pump.

The bioreactor described in the system serves to propagate yeasts for any wine, and it is sufficient simply to activate the first programme.

It is also designed to propagate lactic acid bacteria, based on a programme of lactic acid bacteria propagation where the conditions of revolutions per minute, temperature, oxygenation and time of the process change for 65 hours.

Finally the bioreactor is also designed to carry out autolysis or stirring of fine lee yeasts, based on a yeast autolysis programme.

### DESCRIPTION OF THE DRAWINGS

To complement the description given below and with the aim of helping towards better comprehension of the characteristics of the invention, in accordance with a preferred example of the practical implementation of the same, included as an integral part of said description is a drawing which by way of illustration but not exhaustive, represents the following:

FIG. 1. A schematic upright view of a system of yeast propagation and adaptation for second fermentation in the generation of sparkling wines undertaken in accordance with the aim of the present invention.

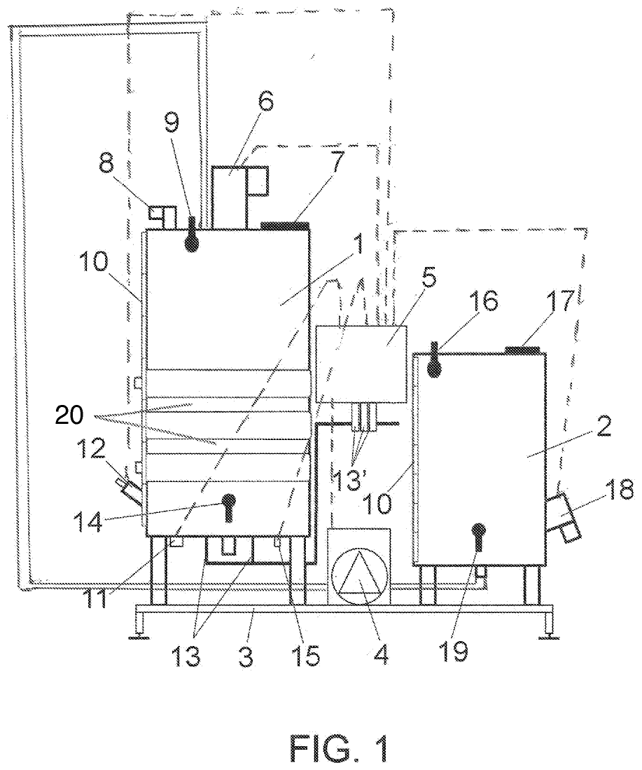

### PREFERRED EMBODIMENT OF THE INVENTION

Apparent in the drawing, it is possible to observe how the recommended device includes a bioreactor (1) in combination with a mixing tank (2), both mounted on a common frame (3) and between them a programmed pump (4), also including a control panel (5) which automates the process, and which includes a tactile screen as seen in the drawing.

The bioreactor (1) includes a motor with stirring blades (6), a folding upper door (7), gas outlet (8), a cleaning ball (9), as well as a measuring scale (10) and cooling chambers which in combination with an internal resistance (11) makes it possible to control the temperature inside said bioreactor (1).

Optionally, it includes an inlet (12) for pH meter.

It also includes an air inlet or diffusors (13) with discharge valves.

In the FIGURE you can see how the device includes sample taking (14) consistent with the bioreactor (1) and a probe (15).

The mixing tank (2) also includes a cleaning ball (16), a folding upper door (17), a stirring motor (18) and sample taking (19) and consistent with the diffusors (13) that determine the entry of air these are also linked to the corresponding air filters (13′).

According to the layout, the process is as follows:

The bioreactor (1) is filled to approximately half of its volume, with the ideal medium for the propagation of yeasts.

The mixing tank (2) is filled with the liquor.

The yeast is activated outside the bioreactor (1) and once activated it is added to the bioreactor so as to start the process with a first controller programme for yeast propagation inside the bioreactor (1), establishing the conditions of r.p.m. of the stirring motor, flow sensor conditions for air inlet and conditions of temperature, so that once the propagation is obtained, the bioreactor enters a second programme combining the conditions previously described, activating the mixing tank (2) and the programmed pump (4) to add the quantities of liquor to the bioreactor (1) itself, a process that lasts 1 day.

Once programme 2 has finalised, it passes to the third programme where the conditions of revolutions, temperature and air flow change, with liquor continuing to enter steadily from the mixing tank (2) to the bioreactor (1), a programme that lasts 1 day.

The yeasts, now propagated and adapted to the ethanol, pH and temperature, having finalised the third programme, can be used for the tirage in the second fermentation, so that during the time the tirage lasts (approximately 5 days) the bioreactor (1) move into a fourth programme where once again the conditions of revolutions, air inlet and temperature change.

What is claimed is:

1. A system for yeast propagation and adaptation for second fermentation in the generation of sparkling wines, characterised as comprising a bioreactor (1) for yeast propagation and a mixing tank (2) containing a liquor, and including a programmed pump (4) for delivering said liquor from the tank (2) to the bioreactor (1) from the mixing tank (2), as well as a programmable automaton with a control panel (5) for process automation connected and driving said programmed pump (4).

2. The system set forth in claim 1, wherein the bioreactor (1) includes a motor with mixing blades (6), as well as air diffusors (13) having corresponding filters (13′), and including a cooling chamber (20) coupled to internal resistances (11) for temperature control inside said bioreactor (1); further including a flow sensor to regulate the entry of filtered air as required by the yeasts to propagate.

3. The system set forth in claim 2, wherein the bioreactor (1), mixing tank (2) and programmed pump (4) are mounted on a common frame (3).

4. The system set forth in claim 3, wherein mixing tank (2) includes a stirring motor (18).

5. The system set forth in claim 4, wherein the bioreactor (1) and the mixing tank (2) include corresponding folding doors (7) and (17) as well as sample takings (14) and (19).

6. The system set forth in claim 5, wherein the bioreactor (1) includes a cleaning ball (9) and a measuring scale (10).

7. The system set forth in claim 5, wherein the mixing tank (2) includes a cleaning ball (16) and a measuring ruler (10).
